(19) United States
(12) Patent Application Publication
(10) Pub. No.: US 2007/0005108 A1
(43) Pub. Date: Jan. 4, 2007

(54) Vacuum holder for juxtaposing body tissues

(76) Inventors: David Simhon, Ramat Efal (IL); Tamar Brosh, Metzada (IL)

Correspondence Address: Mark Friedman;Bill Polkinghorn, 9003 Florin Way, Upper Marlboro, MD, 20772, US

(21) Appl. No.: 10/544,843

(22) PCT Filed: Feb. 10, 2004
(86) PCT No.: PCT/IL04/00129

§ 371 Date: Jul. 24, 2006

Related U.S. Application Data
(60) Provisional application No. 60/445,800, filed on Feb. 10, 2003.

(51) Int. Cl. A61B 17/08

(52) U.S. Cl. 606/216

(57) ABSTRACT

The tissue holding device of the present invention is composed of at least two, substantially hollow, adjustable suction clamps, configured for one-sided clamping, that detachably adhere to the surface of the tissue by way of at least one suction opening in each suction clamp. The suction clamps are mechanically associated by a displacement mechanism. When the suction clamps are drawn toward each other the underlying adherent tissue margins are drawn toward each other. The tissue holding device holds the tissues during a bonding procedure, or the application of tissue bonding material or any other suitable substance to the adjacent tissue margins. When the application process is finished, the suction clamps may be easily removed by releasing the suction force (i.e. negative pressure). The tissue holding device may be used to bring two tissue surfaces together, such as during surgery and for the purpose of wound repair. The suction clamps and the displacement mechanism create a support structure to which additional devices such as, but not limited to, a medical adhesive applicator, a laser, laser optic fibers, and bandage application devices can also be mounted.

### FIELD AND BACKGROUND OF THE INVENTION

The present invention relates to holding body tissue and in particular, to a medical device for temporarily holding body tissue sections in alignment during a bonding procedure in which the tissue sections are bonded together.

The proper alignment of body tissue edges for application of tissue bonding devices or glues is crucial for proper wound healing. Inappropriate tissue alignment may interfere with the normal wound healing process. A device that is capable of achieving and maintaining such proper alignment also frees the surgeon from the need to keep tissue edges in proximity and alignment, thus allowing the surgeon to use both hands freely for other tasks. It also reduces the possibility of adherence of the surgeon's hands or gloves to the underlying tissue bonding material.

A number of devices intended to hold body tissue or organs during medical procedures have been suggested. Some of these are specific to the intention of tissue alignment during connection or bonding procedure such as, for example, gluing, suturing, stapling, clipping, laser welding and laser soldering. Most notable of these devices in relation to the present invention is the device of U.S. Pat. No. 5,611,794 to Sauer et al. Sauer et al. clearly describes an apparatus specific for use with a laser welding device and a clamp mechanism for holding tubular tissues in alignment during the laser welding procedure.

One drawback to the clamp mechanism of Sauer et al., and other such clamps of prior art, is that each arm (jaw) grasps the tissue section from two opposing sides, such as the top and the bottom. In many cases, such as, for example, closing wounds or other procedures where only one surface of each of the tissue sections is exposed or available, the clamp mechanism of Sauer et al. is inoperable. To illustrate this, Sauer et al. suggests that in order to facilitate securement and approximation of tubular tissue sections, an absorbable stent can optionally be utilized. The proximal end of the stent may be inserted into a proximal tubular section prior to clamping. After clamping of the proximal tubular section, the distal tubular section is then pulled over the distal end of the stent. Therefore the sent provides yet another surface between which tissues may be grasped.

Another issue not addressed by the prior art is that of sterilization of the device. Since this issue is not addressed in the documents of prior art, it is reasonable for one of ordinary skill in the art to assume that the entire device is to be sterilized or the device must be disassembled and the pertinent parts are sterilized: The downtime of the device while being sterilized presents a further drawback.

There is therefore a need for a medical device for temporarily holding body tissue sections in alignment during a bonding procedure in which the sections are bonded together, the device grasping each of the tissue sections on only one surface. It would be beneficial if the device were configured with easily detachable grasping elements. It would be of further benefit if the device were partially or entirely configured so as to be disposable.

### SUMMARY OF THE INVENTION

The present invention is a medical device for temporarily holding body tissue sections in alignment.

According to the teachings of the present invention there is provided, a medical device for temporarily juxtaposing edges of two regions of tissue, the device comprising: (a) a first suction clamp configured for one-sided clamping of a first region of tissue; (b) a second suction clamp configured for one-sided clamping of a second region of tissue; and (c) a displacement mechanism mechanically associated with both the first and the second suction clamps and configured to define a path of relative displacement between the first and the second suction clamps for bringing together edges of the first and the second regions of tissue.

According to a further teaching of the present invention, each of the first and the second suction clamps is mechanically associated with the displacement mechanism via at least one pivotal joint, each of the pivotal joints providing at least one degree of pivotal freedom between the suction clamp and the displacement mechanism.

According to a further teaching of the present invention, there is also provided, a locking configuration selectively deployable to lock at least one of the pivotal joints against pivotal motion.

According to a further teaching of the present invention, at least one of the first and the second suction clamps is associated with a suction regulator for controlling a suction force applied by at least one of the first and the second suction clamps.

According to a further teaching of the present invention, at least one of the first and the second suction clamps is associated with a pressure release valve.

According to a further teaching of the present invention, the displacement mechanism includes at least one elongated rod, and wherein at least one of the first and second suction clamps is mounted so as to be slidable along the elongated rod.

According to a further teaching of the present invention, the elongated rod is implemented as a flexible elongated rod such that flexing of the elongated rod facilitates clamping of the first and second suction clamps onto first and second regions of tissue which are non-coplanar.

According to a further teaching of the present invention, the elongated rod is implemented as a flexible elongated rod with memory properties such that when released from the flexing, the elongated rod returns to an original profile.

According to a further teaching of the present invention, at least part of each of the first and second suction clamps is implemented as a detachable clamp contact-surface element coupled to a remaining portion of the device via a quick-release coupling.

According to a further teaching of the present invention, the displacement mechanism includes a pair of elongated arms interconnected at a hinge, wherein the first suction clamp is mechanically associated with a distal portion of a first of the elongated arms, and the second suction clamp is mechanically associated with a distal portion of a second of the elongated arms.

According to a further teaching of the present invention, each of the elongated arms further includes a handle portion extending from the hinge away from the first and second suction clamps so as to form a scissor mechanism.

According to a further teaching of the present invention, the displacement mechanism includes a locking mechanism configured so as to restrict movement of the displacement mechanism.

According to a further teaching of the present invention, distal ends of each of the first and the second suction clamps are connected by mechanical link configured so as to maintain an angular relationship of the first and the second suction clamps.

According to a further teaching of the present invention, the mechanical link includes a locking mechanism configured so as to restrict movement of the first and the second suction clamps.

According to a further teaching of the present invention, there is also provided, a third suction clamp configured for clamping a graft element, the third suction clamp being mechanically associated with the displacement mechanism so as to position a graft element substantially overlying adjacent edges of the first and second regions of tissue.

According to a further teaching of the present invention, the third suction clamp includes a suction regulator for controlling a suction force applied by the third suction clamp.

According to a further teaching of the present invention, the displacement mechanism further includes means for selectively displacing the third suction clamp in a direction substantially perpendicular to the path of relative displacement between the first and the second suction clamps.

According to a further teaching of the present invention, a tissue bonding system is mounted on the displacement mechanism.

According to a further teaching of the present invention, the tissue bonding system is a medical adhesive applicator.

According to a further teaching of the present invention, at least one of the first and the second suction clamps are configured to clamp regions of tissue that are substantially flat.

According to a further teaching of the present invention, at least one of the first and the second suction clamps are configured to clamp regions of tissue that are substantially tubular.

According to a further teaching of the present invention, the displacement mechanism is configured such that a relative height between the first and the second suction clamps is adjustable.

There is also provided according to the teachings of the present invention, a method for temporarily juxtaposing edges of two regions of tissue, the method comprising: (a) providing a displacement mechanism mechanically associated with a first and a second suction clamps configured for one-sided clamping, the displacement mechanism configured to define a path of relative displacement between the first and the second suction clamps for bringing together edges of the first and the second regions of tissue; (b) deploying the first suction clamp on a first region of tissue; (c) deploying the second suction clamp on a second region of tissue; (d) generating suction within the first and the second suction clamps, thereby creating suction bonds between the first suction clamp and the first region of tissue, and the second suction clamp and the second region of tissue; (e) operating the displacement mechanism so as to displace the first and the second suction clamps, thereby bringing together edges of the first and the second regions of tissue.

According to a further teaching of the present invention, there is also provided, providing at least one degree of pivotal freedom between the suction clamp and the displacement mechanism by mechanically associating each of the first and the second suction clamps is with the displacement mechanism via at least one pivotal joint, each of the pivotal joints.

According to a further teaching of the present invention, there is also provided, locking at least one of the pivotal joints against pivotal motion using a selectively deployable locking configuration.

According to a further teaching of the present invention, there is also provided, associating a suction regulator with at least one of the first and the second suction clamps for controlling a suction force applied by at least one of the first and the second suction clamps.

According to a further teaching of the present invention, there is also provided, associating a pressure release valve with at least one of the first and the second suction clamps.

According to a further teaching of the present invention, the displacement mechanism is implemented with at least one elongated rod, and wherein at least one of the first and second suction clamps is mounted so as to be slidable along the elongated rod.

According to a further teaching of the present invention, the elongated rod is implemented as a flexible elongated rod such that flexing of the elongated rod facilitates clamping of the first and second suction clamps onto first and second regions of tissue which are non-coplanar.

According to a further teaching of the present invention, the elongated rod is implemented as a flexible elongated rod with memory properties such that when released from the flexing, the elongated rod returns to an original profile.

According to a further teaching of the present invention, at least part of each of the first and second suction clamps is implemented as a detachable clamp contact-surface element coupled to a remaining portion of the method via a quick-release coupling.

According to a further teaching of the present invention, the displacement mechanism is implemented with a pair of elongated arms interconnected at a hinge, wherein the first suction clamp is mechanically associated with a distal portion of a first of the elongated arms, and the second suction clamp is mechanically associated with a distal portion of a second of the elongated arms.

According to a further teaching of the present invention, each of the elongated arms is implemented with a handle portion extending from the hinge away from the first and second suction clamps so as to form a scissor mechanism.

According to a further teaching of the present invention, the displacement mechanism is implemented with a locking mechanism configured so as to restrict movement of the displacement mechanism.

According to a further teaching of the present invention, there is also provided, connecting distal ends of each of the first and the second suction clamps with a mechanical link configured so as to maintain an angular relationship of the first and the second suction clamps.

According to a further teaching of the present invention, the mechanical link is implemented with a locking mechanism configured so as to restrict distal movement of the first and the second suction clamps.

According to a further teaching of the present invention, there is also provided, mechanically associating a third suction clamp, configured for clamping a graft element, with the displacement mechanism, the third suction clamp being deployable so as to position a graft element substantially overlying adjacent edges of the first and second regions of tissue.

According to a further teaching of the present invention, the third suction clamp is implemented with a suction regulator for controlling a suction force applied by the third suction clamp.

According to a further teaching of the present invention, there is also provided, selectively displacing the third suction clamp in a direction substantially perpendicular to the path of relative displacement between the first and the second suction clamps.

According to a further teaching of the present invention, there is also provided, mounting a tissue bonding system on the displacement mechanism.

According to a further teaching of the present invention, the tissue bonding system is implemented as a medical adhesive applicator.

According to a further teaching of the present invention, there is also provided, configuring the at least one of the first and the second suction clamps for deployment on substantially flat regions of tissue.

According to a further teaching of the present invention, there is also provided, configuring the at least one of the first and the second suction clamps for deployment on substantially tubular regions of tissue.

According to a further teaching of the present invention, there is also provided, adjusting a relative height between the first and the second suction clamps.

### BRIEF DESCRIPTION OF THE DRAWINGS

The invention is herein described, by way of example only, with reference to the accompanying drawings, wherein:

FIG. 1 is an isometric view of a first preferred embodiment of a tissue holding device constructed and operative according to the teachings of the present invention;

FIG. 2 is a partial cut-away isometric view of a second preferred embodiment of a tissue holding device constructed and operative according to the teachings of the present invention;

FIG. 3 is an isometric view from above of a third embodiment of a tissue holding device constructed and operative according to the teachings of the present invention;

FIG. 4 is a top elevation of the embodiment of FIG. 3;

FIG. 5 is an isometric view from below of the embodiment of FIG. 3;

FIG. 6 is a detail of FIG. 5;

FIG. 7 is an isometric view from above of the embodiment of FIG. 3, shown with an additional suction clamp;

FIG. 8 is an isometric view from above of the embodiment of FIG. 3, shown with a distal supplementary mechanical link;

FIG. 9 is a top elevation detail of the embodiment of FIG. 3, showing details of the alignment elements;

FIG. 10 is a front elevation of a first preferred embodiment of a suction clamp for use with tubular tissues constructed and operative according to the teachings of the present invention;

FIG. 11 is an isometric view of the suction clamp of FIG. 10;

FIG. 12 is an isometric view of two suction clamps of FIG. 10 used to hold two sections of tubular tissue;

FIG. 13 is front elevation of a second preferred embodiment of a suction clamp for use with tubular tissues constructed and operative according to the teachings of the present invention, shown here in an open deployment; and

FIG. 14 is a front elevation of the suction clamp of FIG. 13 shown in a closed deployment.

### DESCRIPTION OF THE PREFERRED EMBODIMENTS

The present invention is a medical device for temporarily holding body tissue sections in alignment.

The principles and operation of a medical device for temporarily holding body tissue sections in alignment according to the present invention may be better understood with reference to the drawings and the accompanying description.

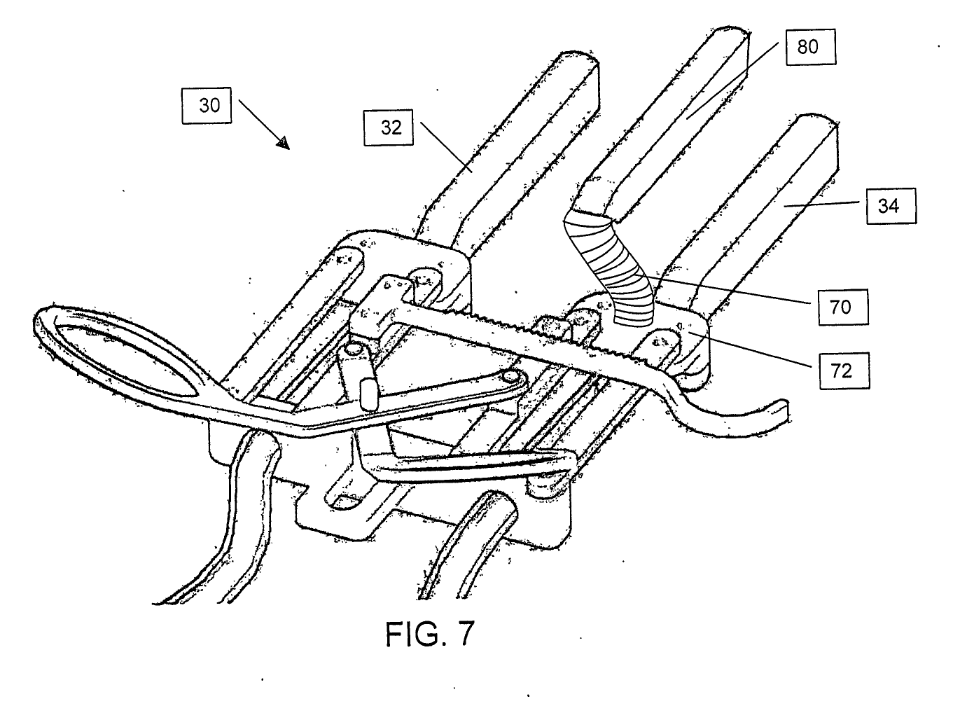

By way of introduction, a tissue holding device of the present invention helps the surgeon achieve and maintain proper tissue edge alignment and proximity for as long as needed during a tissue bonding procedure. An automatic tissue bonding apparatus, such as, but not limited to, a laser, laser optic fibers, or medical adhesive applicator can also be mounted on the suggested frame of substantially any embodiment of the present invention for automatic tissue bonding. Any of several bandage application devices, as are known in the art, may also be connected to the frame of the present invention so as to facilitate bandage application after the bonding procedure is finished and before the tissue holding device is removed. Such bandage application while the tissue sections are supported by the tissue holding device helps support the bonded tissue edges in a manner similar to the support given by the device even after the tissue is no longer supported by the tissue holding device. Further, additional suction clamps may be attached so as to hold, by non-limiting example, an additional piece of grafting material, as in a grafting procedure. The grafting material may be one of several different materials, such as, but not limited to, natural tissue, tissue cultures and biological or synthetic grafting material. The schematic illustration of FIG. 7 is a non-limiting suggestion for the attachment of an additional suction clamp 80 for holding an additional piece of grafting material, which is deployed above the working area between the suction clamps 32 and 34, to the tissue holding device 30. As illustrated here, the attachment bracket 70 is, by non-limiting example, a flexible metal tube, such as is commonly used on microphone stands, that has plastic properties and may therefore be bent so as to position the additional suction clamp 80 substantially wherever necessary. Other such additional devices may include, but not be limited to, an automatic tissue bonding apparatus and a bandage application device, as mentioned above. When the additional device requires a fixed position, bracket 70 may be configured so as to be substantially ridged and fixedly attached to attachment flange 72. As discussed above, when the additional device requires repositioning during use, bracket 70 may be flexible, or alternatively, movably attached, such as, but not limited to hinged, rotatable, and slideable, to attachment flange 72. It should be noted that when the additional device is a suction device, such as, but not limited to, the additional suction clamp described above, a vacuum regulator and an emergency pressure release mechanism, as are discussed below, may also be associated with the vacuum elements of the additional device.

It should be noted that the tissue holding device of the present invention may also be used in conjunction with, but not be limited to, gluing, suturing, stapling, clipping, laser welding and laser soldering.

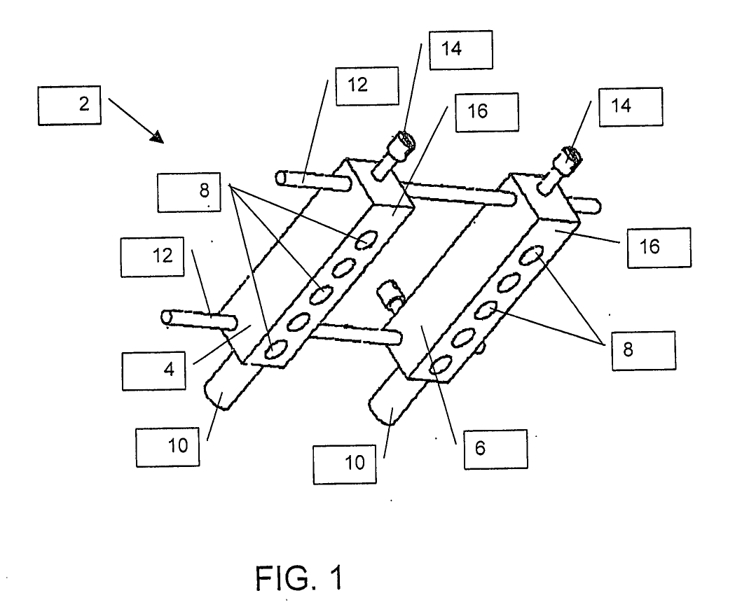

In general, as illustrated in a first preferred embodiment of FIG. 1, the tissue holding device 2 of the present invention is composed of at least two, substantially hollow, adjustable suction clamps 4 and 6 having a substantially flat profile. The suction clamps are configured for one-sided clamping of the tissue. That is, each suction clamp element is operative to grip the adjacent tissue without requiring an opposing element against which to apply clamping pressure. The suction clamps detachably adhere to the surface of substantially flat tissue by way of at least one suction opening 8 in each suction clamp. Suction is created by generating negative pressure, i.e. pressure below atmospheric, within the suction clamps. In a preferred embodiment of the present invention, negative pressure in the range of 100-800 millibars below atmospheric pressure is used. This range, however, is not intended as a limitation, and substantially any amount of negative pressure required may be generated with in the suction clamps. When the suction openings are placed against the surface of the tissue the suction creates one-sided clamping of the tissue. That is, a suction bond is created between the suction clamp and one-side of the tissue. Therefore, the phrase “substantially flat tissue” is used herein to refer tissue having a surface suited for clamping with a suction clamp having a flat profile and on which a suction bond may be created. That is, the tissue is flat or has a radius of curvature not more than a few centimeters so that the region clamped by the suction clamps typically does not turn through more than about 30 degrees. In the embodiment of FIG. 1, each of the suction clamps includes a tube attachment extension 10 whereby a tube may be used to establish fluid communication between each suction clamp and a vacuum generation source.

The suction clamps are mechanically associated by a displacement mechanism. In one preferred implementation as shown here, the displacement mechanism includes at least one elongated rod 12, which may be of substantially any cross-sectional contour. The suction clamps 4 and 6 and elongated rod 12, or rods depending on the particular application, create a framework around the tissue edges. When the suction clamps 4 and 6 are drawn toward each other by sliding one or both of them along the elongated rods 12, the underlying adherent tissue margins are drawn toward each other. The suction clamps 4 and 6, may be locked in place on the elongated rods 12, by, for example, set screws 14. Once the tissue margins are aligned, positioning of the tissue edges may be finalized using forceps. The tissue holding device 2 holds the tissues during the application of tissue bonding material or any other suitable substance to the adjacent tissue margins. When the application process is finished, the suction clamps 4 and 6 may be easily removed by releasing the suction, i.e. negative pressure. The tissue holding device 2 may be used externally to bring two skin surfaces together, such as, but not limited to, following surgery, and wound repair.

It should be noted that alternatively, the tissue holding device of the present invention may also be configured so as to be useable internally, such as, but not limited to, procedures on an internal organ. The tissue holding device may also be inserted into a natural or temporarily-generated body cavity via any opening for minimally invasive surgery (endoscopy) purposes.

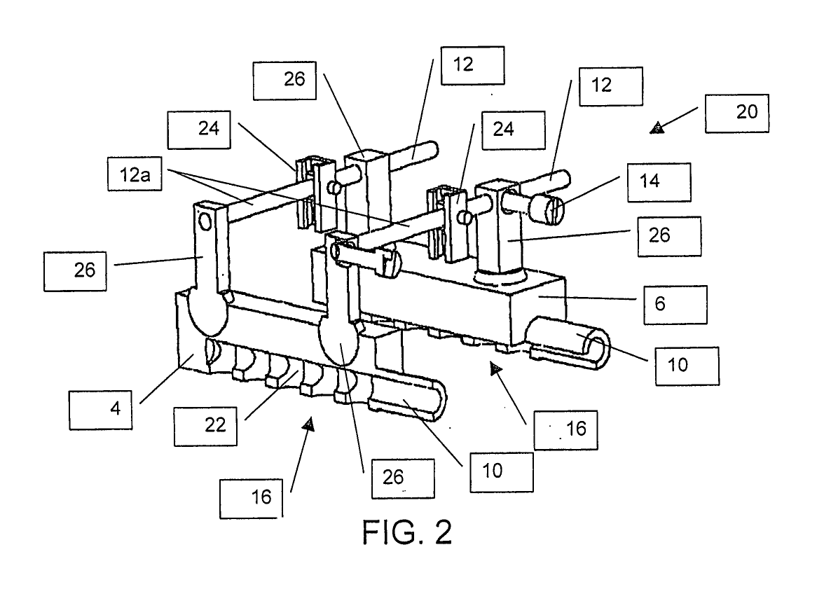

FIG. 2 illustrates a second preferred embodiment of the tissue holding device 20 of the present invention. The hollow portion 22 of the suction clamps 4 and 6 is visible in the cut-way portion of suction clamp 4. In this embodiment, the elongated rods 12 include fixedly attached height adjustment brackets 24 that are configured to allow the height of suction clamp 6 relative to suction clamp 4 to be adjusted. As illustrated here, rods 12a are slideably engaged in the height adjustment bracket and the relative height of suction clamps 4 and 6 adjusted. Once the desired height differential is achieved, the engaged ends of rods 12a are locked in the height adjustment brackets 24. The elongated rods 12 are attached to the suction clamps 4 and 6 by articulation extensions that attach to the suction clamps 4 and 6 in, for example, a ball and socket configuration that allows the angle of alignment of the suction clamps in relation to each other to be varied. That is, in the embodiment of FIG. 1 the contact surface 16 of the suction clamps 4 and 6 are coplanar. The articulation extensions 26 of FIG. 2 allow the contact surface 16 of the suction clamps 4 and 6 to be aligned so as to be either coplanar or non-coplanar. Here too, set screws 14 may be used to lock the position of the suction clamps 4 and 6.

In some applications it may be desirable to provide flexible elongated rods 12 in order to facilitate bringing the contact surfaces 16 of the suction clamps 4 and 6 into contact with the surfaces of the tissues to which they are to adhere. In such applications, the elongated rods 12 may be configured from flexible materials having plastic or inelastic properties allowing them to be bent to shape. Alternatively, the elongated rods 12 may be configured from materials with elastic properties so as to be bendable in order to achieve adhesion to the tissue and then return to the original shape.

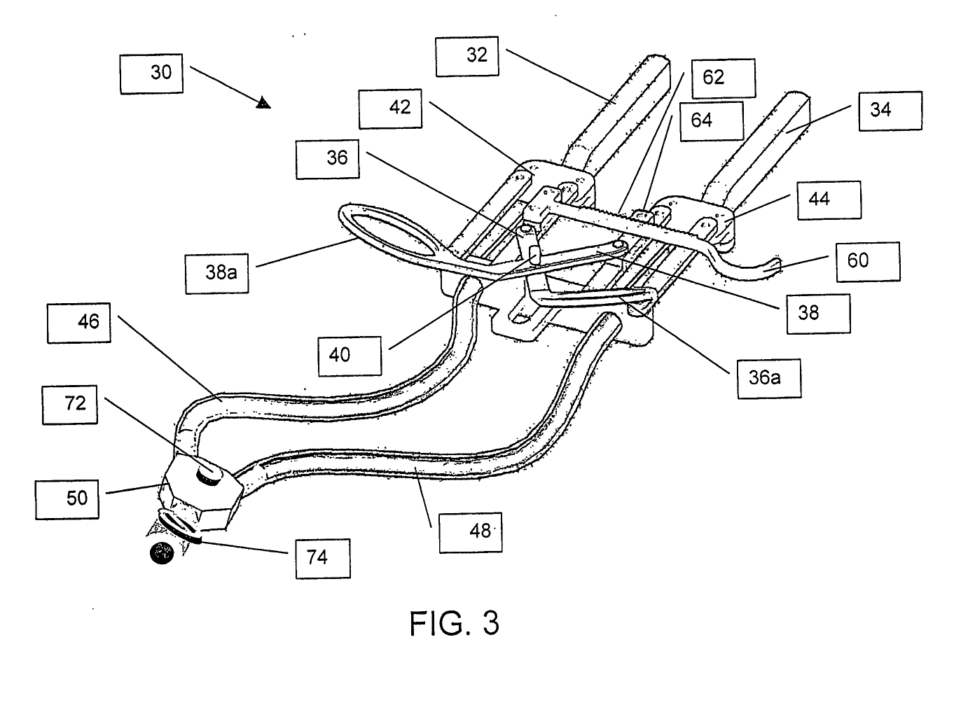

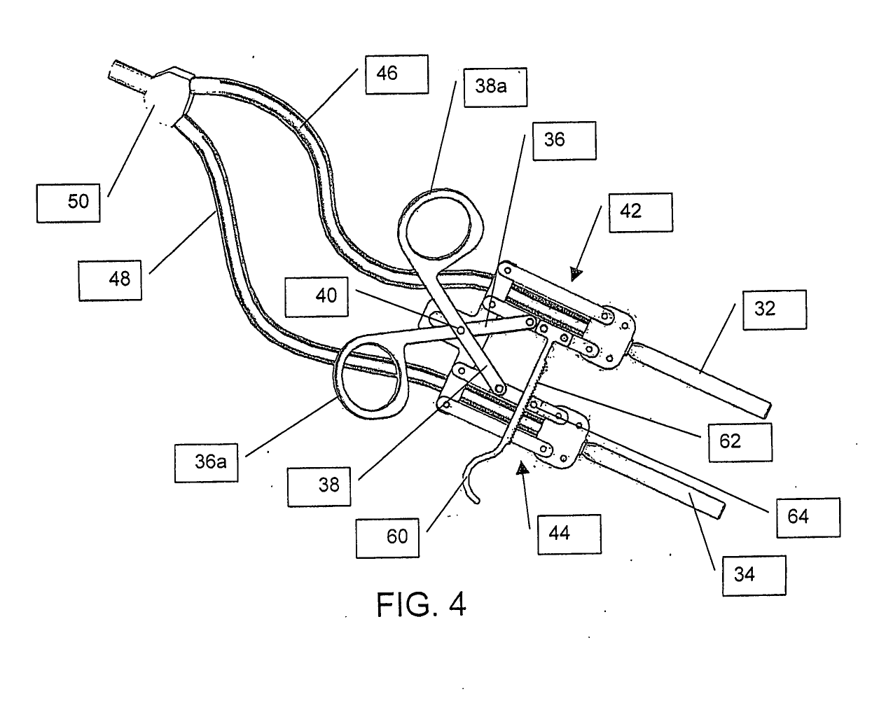

FIGS. 3-6 illustrate a third preferred embodiment of the present invention, generally referred to as 30, in which the displacement mechanism includes a pair of elongated arms 36 and 38 interconnected at a hinge 40. As shown in the illustration here, the elongated arms 36 and 38 extend beyond the hinge 40 into handles 36a and 38a respectively, thereby forming a scissor mechanism such that suction clamps 32 and 34 are positioned by rotation of the elongated arms 36 and 38 about the hinge 40.

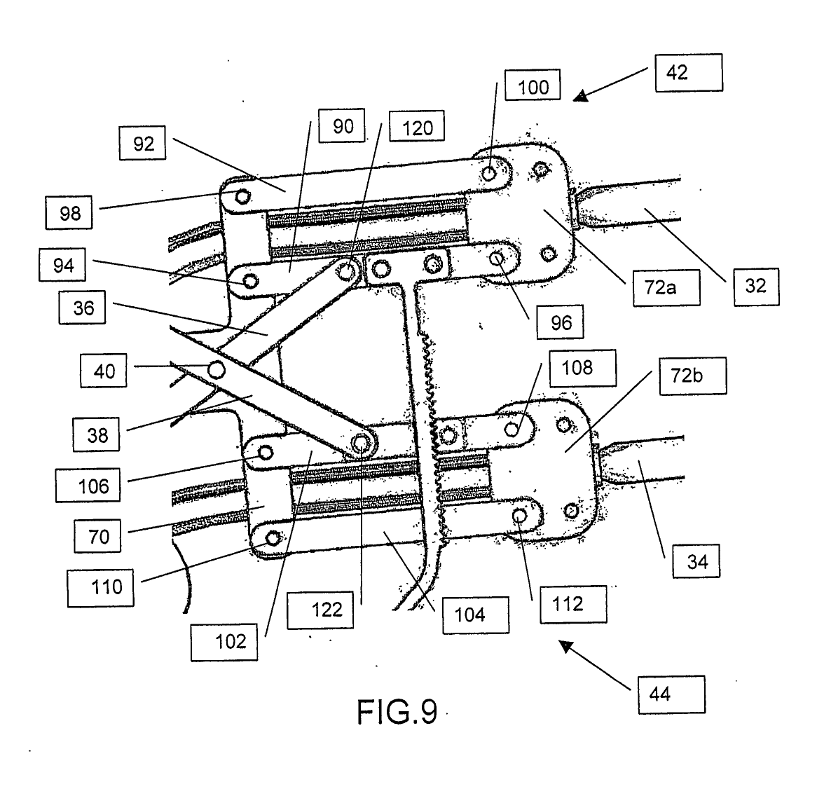

In this embodiment, the displacement mechanism also includes a pair of alignment elements 42 and 44, which are configured to maintain the angular alignment of the suction clamps 32 and 34. As seen in detail in FIG. 9, alignment element 42 includes two parallel linear bars 90 and 92 that are rotatably attached to the cross bar 70 by hinge pins 94 and 98, respectively, and to the top of attachment flange 72a by hinge pins 96 and 100 respectively. Linear bar 90 is also rotatably attached to elongated arm 36 by hinge pin 120. Thusly configured, the alignment of the cross bar 70 and the attachment flange 72 is maintained as the position of the attachment flange 72a is changed. Similarly, alignment element 44 includes two parallel linear bars 102 and 104 that are rotatably attached to the cross bar 70 by hinge pins 106 and 110, respectively, and to the top of attachment flange 72b by hinge pins 108 and 112, respectively. Linear bar 90 is also rotatably attached to elongated arm 38 by hinge pin 122. It will be readily appreciated that as elongated arms 36 and 38 are rotated about hinge pin 40 in a scissor type motion the position of each of the alignment elements 42 and 44 will correspondingly change as they rotate about their respective hinge pins, but the angle of alignment between the cross bar 70 and each of the attachment flanges 72a and 72b will remain substantially constant. Preferably, the suction clamps 32 and 34 are aligned so as to be parallel; however, substantially any angle of alignment is within the scope of the present invention.

A further optional structural feature illustrated here is the joining of vacuum tubes 46 and 48 at tube coupling element 50, thereby reducing the number of tubes required to connect the tissue holding device to the vacuum source necessary to operate the suction clamps 32 and 34.

The proper position of the suction clamps 32 and 34 in this embodiment is locked by the locking arm 60. As illustrated here, the locking arm 60 is mounted on alignment element 42 and has a serrated edge 62 configured to engage the serration of the locking block 64 mounted on alignment element 44. The locking arm 60 is preferably configured from material with spring properties so as to be biased toward the locking block 62. Alternatively, the locking arm 60 may be mechanically biased toward the locking block 64 by, for example, a spring. It should be noted that the locking arm 60 and locking block 64 may alternatively be appropriately mounted on the elongated arms 36 and 38 of the scissor mechanism, or substantially any appropriate points on the displacement mechanism. Further, substantially any locking mechanism may be used to lock the position of the suction clamps once they are positioned.

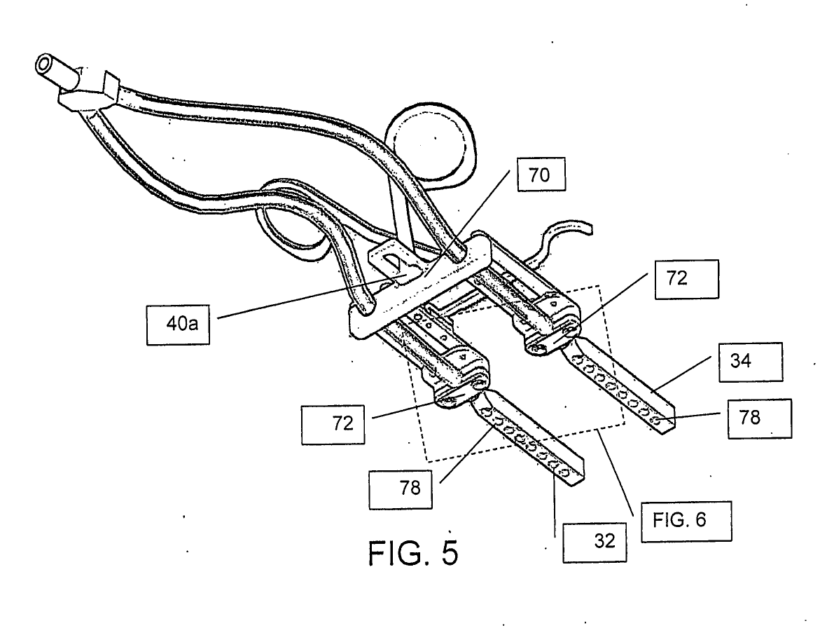

As seen in better detail in FIG. 5, the axle 40a of hinge 40 extends downward and attaches to the cross bar 70 of the displacement mechanism, thereby facilitating substantially equal displacement of the suction clamps as the scissor mechanism is operated. The suction openings 78 are also clearly visible from this angle.

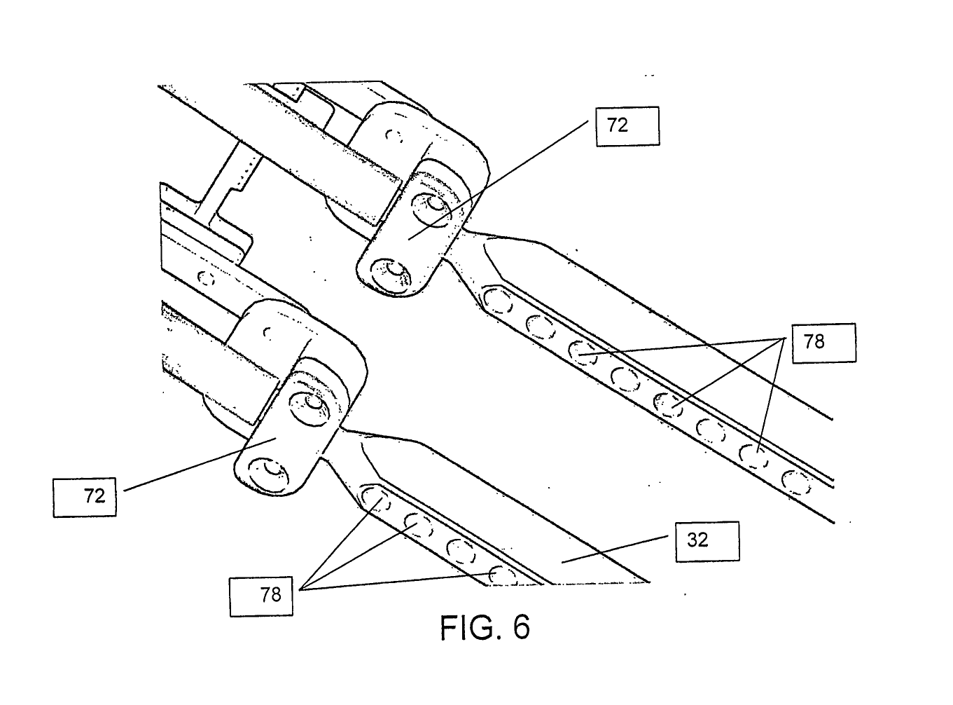

The detail of FIG. 6 illustrates the attachment flanges 72 used to attach the suction clamps to the displacement mechanism. The attachment flanges 72 may be configured in any one or combination of several configurations, including, but not limited to:
1. The attachment flanges 72 being solidly affixed such that the suction clamps 32 and 34 are permanently attached to the displacement mechanism and are non-rotatable.
2. The attachment flanges 72 being releasably affixed such that the suction clamps 32 and 34 are releasably attached to the displacement mechanism. Affixation of this sort may be by use of screws or nuts and bolts, for non-limiting example.
3. The attachment flanges 72 being releasably affixed such that the suction clamps 32 and 34 are rotatable.
4. The attachment flanges 72 being releasably affixed so as to act as “quick release” elements such that the suction clamps 32 and 34 are quickly and easily removable from the displacement mechanism.

Although not illustrated herein, either one or both alignment elements 42 and 44 may be configured so as to allow for the adjustment of the relative height of suction clamps 32 and 34 to one another, as was discuss above with regard to FIG. 2. This may be accomplished by, but not be limited to, articulation of the alignment elements, sliding brackets attached to the attachment flanges 72, and substantially any other height adjustment mechanism know that is adaptable to the elements of the present invention.

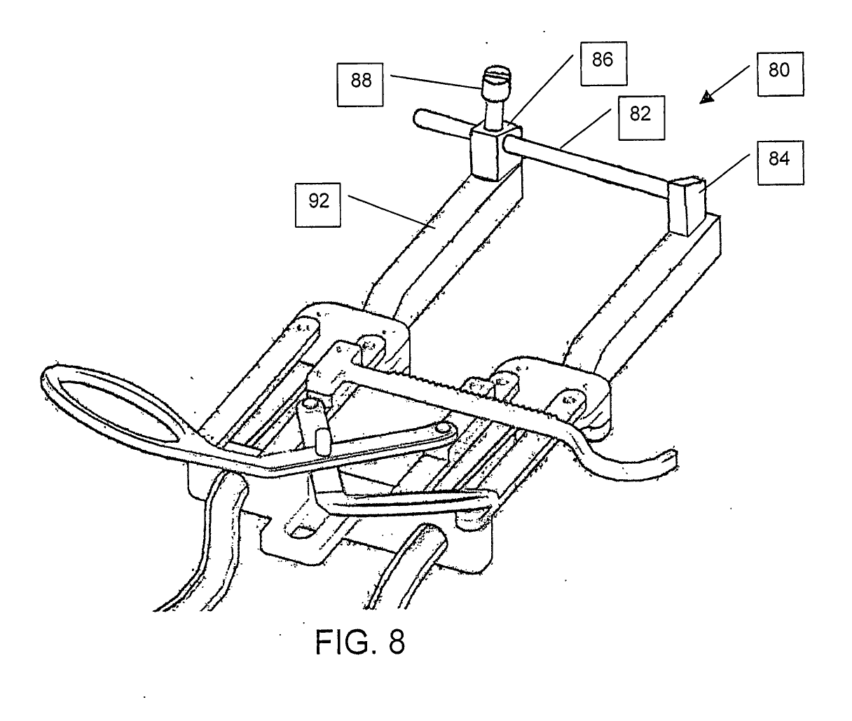

It should be noted that for applications requiring the use of long suction clamps such that the angle of alignment of the suction clamps may not be maintained by the displacement mechanism that is attached to the proximal end of the suction clamps, a supplementary mechanical link may be attached at the distal end of the suction clamps, such as the non-limiting example of 80 in FIG. 8. The supplementary mechanical link illustrated here includes a link bar 82 that is fixedly attached at one end to a raised portion of the distal end of suction clamp 90. The link bar 82 is slidingly interconnected to a raised portion 86 of suction clamp 92. The raised portion 86 includes a locking screw 84 configured to lock the position of the link bar 82. Therefore, during positing of the suction clamps 90 and 92, the link bar 82 slides within raised portion 86, and once the suction clamps 90 and 92 are positioned, the locking screw 84 is tightened so as to lock the position of the link bar 82, thereby maintaining the distance between, and thus the alignment of, the suction clamps 90 and 92. The raised portions 84 and 86 may be integrally formed on the respective suction clamps or they may be separate elements that are attached to the suction clamps.

With regard to substantially all of the embodiments possible according to the teaching of the present invention, it should be noted that at least a portion of the section clamps may be configured so as to be disposable. The disposable portion may be attached so as to be quickly and easily detachable, such as by a quick release mechanism. Alternatively, each of the suction clamps may be covered by disposable perforated sleeves; this is particularly applicable to the embodiment of FIG. 3. It will be appreciated, then, that any appropriate part or the entire device may be disposable.

As illustrated in FIG. 3, a vacuum regulator 74 may be associated with the tissue holding device so as to regulate the suction force, i.e. negative pressure, within the suction clamps. An emergency pressure release mechanism 72 may also be associated with any of the vacuum elements of the tissue holding device; here it is incorporated into the tube coupling element 50. The emergency pressure release may be actuated either automatically or manually, or a combination of the two.

With regard to the suction clamps, they may be of substantially any length and any cross-sectional contour. They may be manufactured in different fixed lengths. Alternatively, the suction clamps may be manufactured so as to be “cut to length” by the end user. This may be accomplished by, but not limited to, supplying extended lengths of suction clamp having a tube attachment extension on one end, such that the suction clamp is cut to a desired length, and a plug, for example, is inserted into the open (cut off) end. The suction openings may be configured as, but not limited to, holes in the contact surface of the suction clamps, or as suction pads or cups extending from the contact surface. The suction openings may be of substantially any size or shape. Further, the size and shape of the suction openings on the contact surface may be uniform or of mixed sizes, shapes or both.

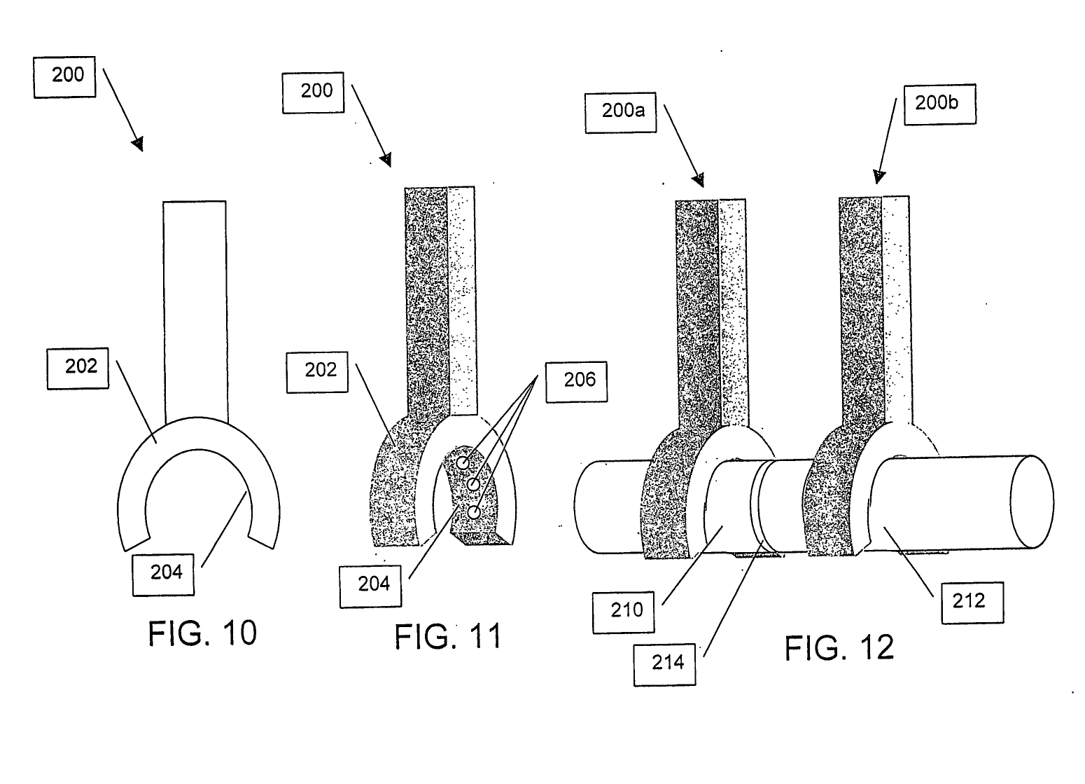

FIGS. 10-14 illustrate two alternative, non-limiting, examples of suction clamp embodiments of the present invention configured for use With tubular tissue, such as, but not limited to, blood vessels. Either of these embodiments may be associated with any of the above described displacement mechanisms to provide the movement necessary to bring tissue edges together for a bonding procedure.

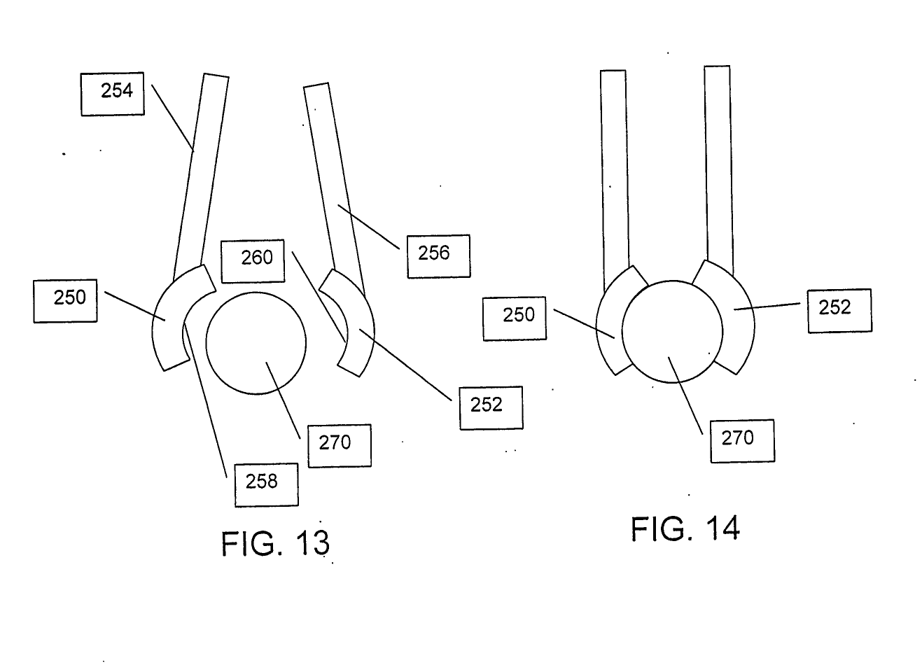

A first preferred embodiment of a suction clamp 200, for use with tubular tissues, is configured as a single at least partially-circular clamp element 202 having a contact surface 204 on its inner surface. That is to say, the contour of the contact surface 204 is shaped so as to include at least part of the circumference of a cylinder. The total proportion of the circumference of the cylinder circumscribed by the suction clamp, either as a single clamp element (FIG. 10), or by the two or more clamp elements considered together (FIG. 13), may range from as little as 30 degrees up to 360 degrees. Most preferably, the circumference of the cylinder circumscribed by a single clamp element is in the range of 180 degrees to 240 degrees. For two or more clamps considered together the preferred total range is between 150 degrees and 200 degrees. The contact surface 204 is configured with suction openings 206 as described above. Thus, when deployed so as to partially circumscribe the tubular tissue and suction is generated within the clamp element, a suction bond is created between the contact surface and the surface of the tissue.

As illustrated in FIG. 12, a pair of such suction clamp elements 200a and 200b is used to bring the edges of tissues 210 and 212 together in region 214.

Alternatively, the single clamp element 200 may be configured as two substantially opposing clamp elements 250 and 252 that may be alternated between an open deployment, as illustrated by the second preferred embodiment of a suction clamp for use with tubular tissues in FIG. 13, and a closed deployment as illustrated in FIG. 14. As shown here, clamp elements 250 and 25 are deployed at distal ends of rods 254 and 256, respectively. Contact surfaces 258 and 260 are each configured with suction openings (not shown). Therefore, when clamp elements 250 and 252 are brought to a closed deployment with a tubular tissue 270 therebetween, and suction is generated within the each of the clamp elements 250 and 252, a suction bond is created between the contact surfaces 258 and 260, and the surface of the tissue. In order to perform the procedure illustrated in FIG. 12 with this embodiment of the present invention, it would be necessary to use two sets of opposing clamp elements, each set clamping a different tissue section.

The benefit of using the section clamps configured for use with tubular tissue during internal medical procedures will be readily appreciated by one of ordinary skill in the art. An appropriate displacement mechanism for such internal deployment will also be readily apparent to one of ordinary skill in the art, and may be configured from, by non-limiting example, a single interconnecting rod upon which at least one of the suction clamps may be displaced. It will also be understood that the elements of the device may be inserted into a body cavity through a single opening or multiple openings.

It will be appreciated that the above descriptions are intended only to serve as examples, and that many other embodiments are possible within the scope of the present invention as defined in the appended claims.

What is claimed is:

1. A medical device for temporarily juxtaposing edges of two regions of tissue, the device comprising:
(a) a first suction clamp configured for one-sided clamping of a first region of tissue;
(b) a second suction clamp configured for one-sided clamping of a second region of tissue; and
(c) a displacement mechanism mechanically associated with both said first and said second suction clamps and configured to define a path of relative displacement between said first and said second suction clamps for bringing together edges of said first and said second regions of tissue.

2. The device of claim 1, wherein each of said first and said second suction clamps is mechanically associated with said displacement mechanism via at least one pivotal joint, each of said pivotal joints providing at least one degree of pivotal freedom between said suction clamp and said displacement mechanism.

3. The device of claim 2, further comprising a locking configuration selectively deployable to lock at least one of said pivotal joints against pivotal motion.

4. The device of claim 1, wherein at least one of said first and said second suction clamps is associated with a suction regulator for controlling a suction force applied by at least one of said first and said second suction clamps.

5. The device of claim 1, wherein at least one of said first and said second suction clamps is associated with a pressure release valve.

6. The device of claim 1, wherein said displacement mechanism includes at least one elongated rod, and wherein at least one of said first and second suction clamps is mounted so as to be slidable along said elongated rod.

7. The device of claim 6, wherein said elongated rod is implemented as a flexible elongated rod such that flexing of said elongated rod facilitates clamping of said first and second suction clamps onto first and second regions of tissue which are non-coplanar.

8. The device of claim 7, wherein said elongated rod is implemented as a flexible elongated rod with memory properties such that when released from said flexing, said elongated rod returns to an original profile.

9. The device of claim 1, wherein at least part of each of said first and second suction clamps is implemented as a detachable clamp contact-surface element coupled to a remaining portion of the device via a quick-release coupling.

10. The device of claim 1, wherein said displacement mechanism includes a pair of elongated arms interconnected at a hinge, wherein said first suction clamp is mechanically associated with a distal portion of a first of said elongated arms, and said second suction clamp is mechanically associated with a distal portion of a second of said elongated arms.

11. The device of claim 10, wherein each of said elongated arms further includes a handle portion extending from said hinge away from said first and second suction clamps so as to form a scissor mechanism.

12. The device of claim 10, wherein said displacement mechanism includes a locking mechanism configured so as to restrict movement of said displacement mechanism.

13. The device of claim 10, wherein distal ends of each of said first and said second suction clamps are connected by mechanical link configured so as to maintain an angular relationship of said first and said second suction clamps.

14. The device of claim 13, wherein said mechanical link includes a locking mechanism configured so as to restrict movement of said first and said second suction clamps.

15. The device of claim 1, further comprising a third suction clamp configured for clamping a graft element, said third suction clamp being mechanically associated with said displacement mechanism so as to position a graft element substantially overlying adjacent edges of the first and second regions of tissue.

16. The device of claim 15, wherein said third suction clamp includes a suction regulator for controlling a suction force applied by said third suction clamp.

17. The device of claim 16, wherein said displacement mechanism further includes means for selectively displacing said third suction clamp in a direction substantially perpendicular to said path of relative displacement between said first and said second suction clamps.

18. The device of claim 1, wherein a tissue bonding system is mounted on said displacement mechanism.

19. The device of claim 18, wherein said tissue bonding system is a medical adhesive applicator.

20. The device of claim 1, wherein said at least one of said first and said second suction clamps are configured to clamp regions of tissue that are substantially flat.

21. The device of claim 1, wherein said at least one of said first and said second suction clamps are configured to clamp regions of tissue that are substantially tubular.

22. The device of claim 1, wherein said displacement mechanism is configured such that a relative height between said first and said second suction clamps is adjustable.

23. A method for temporarily juxtaposing edges of two regions of tissue, the method comprising:
(a) providing a displacement mechanism mechanically associated with a first and a second suction clamps configured for one-sided clamping, said displacement mechanism configured to define a path of relative displacement between said first and said second suction clamps for bringing together edges of said first and said second regions of tissue;
(b) deploying said first suction clamp on a first region of tissue;
(c) deploying said second suction clamp on a second region of tissue;
(d) generating suction within said first and said second suction clamps, thereby creating suction bonds between said first suction clamp and said first region of tissue, and said second suction clamp and said second region of tissue;
(e) operating said displacement mechanism so as to displace said first and said second suction clamps, thereby bringing together edges of said first and said second regions of tissue.

24. The method of claim 23, further including providing at least one degree of pivotal freedom between said suction clamp and said displacement mechanism by mechanically associating each of said first and said second suction clamps is with said displacement mechanism via at least one pivotal joint, each of said pivotal joints.

25. The method of claim 24, further comprising locking at least one of said pivotal joints against pivotal motion using a selectively deployable locking configuration.

26. The method of claim 23, further including associating a suction regulator with at least one of said first and said second suction clamps for controlling a suction force applied by at least one of said first and said second suction clamps.

27. The method of claim 23, further including associating a pressure release valve with at least one of said first and said second suction clamps.

28. The method of claim 1, wherein said displacement mechanism is implemented with at least one elongated rod, and wherein at least one of said first and second suction clamps is mounted so as to be slidable along said elongated rod.

29. The method of claim 28, wherein said elongated rod is implemented as a flexible elongated rod such that flexing of said elongated rod facilitates clamping of said first and second suction clamps onto first and second regions of tissue which are non-coplanar.

30. The method of claim 29, wherein said elongated rod is implemented as a flexible elongated rod with memory properties such that when released from said flexing, said elongated rod returns to an original profile.

31. The method of claim 23, wherein at least part of each of said first and second suction clamps is implemented as a detachable clamp contact-surface element coupled to a remaining portion of the method via a quick-release coupling.

32. The method of claim 23, wherein said displacement mechanism is implemented with a pair of elongated arms interconnected at a hinge, wherein said first suction clamp is mechanically associated with a distal portion of a first of said elongated arms, and said second suction clamp is mechanically associated with a distal portion of a second of said elongated arms.

33. The method of claim 32, wherein each of said elongated arms is implemented with a handle portion extending from said hinge away from said first and second suction clamps so as to form a scissor mechanism.

34. The method of claim 32, wherein said displacement mechanism is implemented with a locking mechanism configured so as to restrict distal movement of said displacement mechanism.

35. The method of claim 32, further including connecting distal ends of each of said first and said second suction clamps with a mechanical link configured so as to maintain an angular relationship of said first and said second suction clamps.

36. The method of claim 35, wherein said mechanical link is implemented with a locking mechanism configured so as to restrict movement of said first and said second suction clamps.

37. The method of claim 23, further comprising mechanically associating a third suction clamp, configured for clamping a graft element, with said displacement mechanism, said third suction clamp being deployable so as to position a graft element substantially overlying adjacent edges of the first and second regions of tissue.

38. The method of claim 37, wherein said third suction clamp is implemented with a suction regulator for controlling a suction force applied by said third suction clamp.

39. The method of claim 38 further including selectively displacing said third suction clamp in a direction substantially perpendicular to said path of relative displacement between said first and said second suction clamps.

40. The method of claim 23, further including mounting a tissue bonding system on said displacement mechanism.

41. The method of claim 40, wherein said tissue bonding system is implemented as a medical adhesive applicator.

42. The method of claim 23, further including configuring said at least one of said first and said second suction clamps for deployment on substantially flat regions of tissue.

43. The method of claim 23, further including configuring said at least one of said first and said second suction clamps for deployment on substantially tubular regions of tissue.

44. The method of claim 23, further including adjusting a relative height between said first and said second suction clamps.
